(19) United States
(12) Patent Application Publication
(10) Pub. No.: US 2009/0026101 A1
(43) Pub. Date: Jan. 29, 2009

(54) Disposable scented sanitary bag

(76) Inventor: Zellestine C. Hicks, Jonesboro, GA (US)

Correspondence Address: Zellestine C. Hicks, 8656 Collins Dr., Jonesboro, GA, 30236, US

(21) Appl. No.: 11/880,641

(22) Filed: Jul. 24, 2007

(51) Int. Cl. A61B 19/00; B65D 30/20

(52) U.S. Cl. 206/438; 383/120

(57) ABSTRACT

The disposable scented sanitary bag will bring a newly designed way to dispose of sanitary napkins. The sanitary bag has drawstring sides to keep odor in with a wonderful scented strip for freshness.

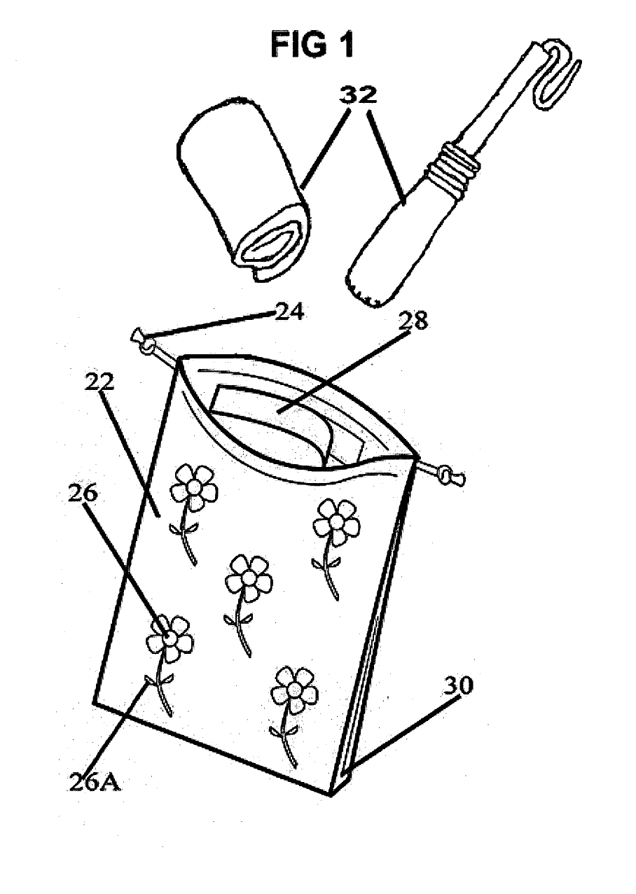

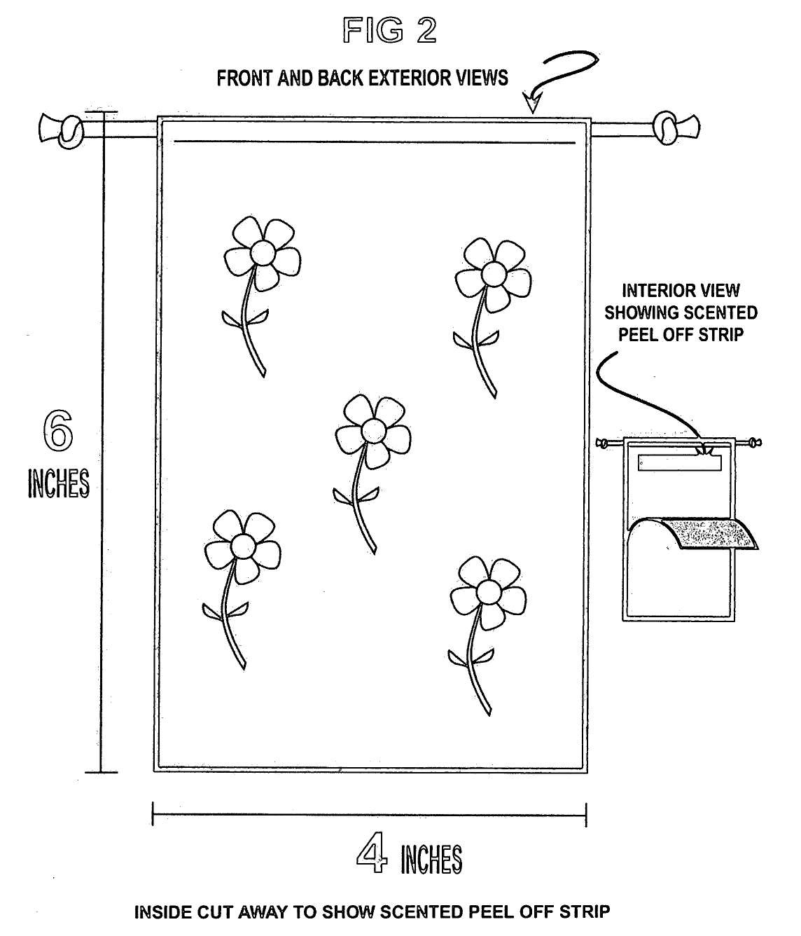

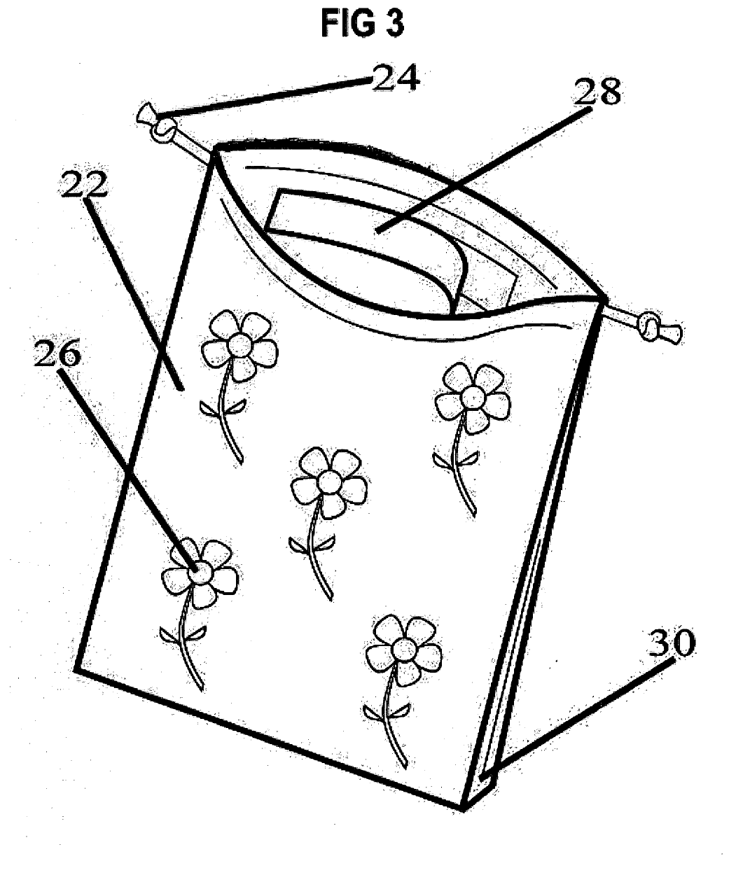

The sanitary bag is a 4 by 6 poly bag with drawstring at each end for closing. The sanitary bag also has a scented strip inside for freshness. The sanitary bag is for the disposal of sanitary napkins and tampons. The bag will be made of dye cut plastic and cotton drawstring.

The scented strip will be made from a special type paper that conseals floral scented fragrances that release when scented strip is peeled away.

1. I claim a plastic sanitary bag with a scented peel strip inside of the bag. The sanitary bag having daisies on the front as well as the rear of the bag. The bag will have gussets on either side useful for larger sanitary napkins and tampons. This bag will have a drawstring on either side to close the bag.
